(19) United States
(12) Patent Application Publication
(10) Pub. No.: US 2019/0385731 A1
(43) Pub. Date: Dec. 19, 2019

(54) INTERACTIVE AND DYNAMIC APPLICATION FOR IMPROVING EMERGENCY ROOM EFFICIENCY AND METHOD OF USE

(71) Applicant: NAVIGATER, LLC, Cape Elizabeth, ME (US)

(72) Inventors: Samir Haydar, Cape Elizabeth, ME (US); Mehmet Kazgan, Denver, CO (US); Fuad Haydar, Fayetteville, AR (US); Tania Strout, Lisbon Falls, ME (US); Burak Cetin, Denver, CO (US); Mike Yunes, South Portland, ME (US)

(21) Appl. No.: 16/446,126

(22) Filed: Jun. 19, 2019

Related U.S. Application Data
(60) Provisional application No. 62/686,998, filed on Jun. 19, 2018.

(51) Int. Cl. G16H 40/20; G16H 40/40; G16H 80/00; G16H 10/60; A61B 5/00

(52) U.S. Cl. CPC G16H 40/20; G16H 40/40; A61B 5/0002; G16H 10/60; G16H 80/00

(57) ABSTRACT

A system including a mobile application and methods of interacting with application users to transmit targeted, individually-curated information based on a user's needs and a calculated experience value index score. The mobile application provides a transparent and secure platform through which authorized users can receive relevant information and provide feedback regarding satisfaction levels. The mobile application receives user feedback and determines the proper information to send to individual application users, the information curated to individual user needs. Observed outcomes scores are compared with expected outcomes scores to calculate an experience value index score, which is analyzed and compared with predetermined threshold values. If a user's experience value index score is above a predetermined threshold, the system transmits routine information to the user. If the user's experience value index score is below the predetermined threshold, the system transmits interactive information to the user to improve user satisfaction.

### CROSS-REFERENCE TO RELATED APPLICATIONS

This nonprovisional patent application is a continuation of and claims the benefit of provisional application No. 62/686,998, entitled “Interactive and dynamic application for improving emergency room efficiency and method of use,” filed on Jun. 19, 2018, by the same inventors.

### BACKGROUND OF THE INVENTION

### 1. Field of the Invention

This invention relates, generally, to applications and methods designed to improve emergency room efficiencies. More specifically, it relates to an interactive and dynamic application that uses communicative technologies, such as mobile networks, to gather patient data and send content and updates based on the gathered patient data, allowing patients to receive real-time updates and targeted content based on the emergency room experience.

### 2. Brief Description of the Prior Art

Patients in emergency room settings often experience the stressful situation of being unaware of their wait times before seeing a healthcare provider. In some situations, the patient is not told of a wait time upon checking into a clinic. In other situations, the patient may be told of a wait time, but another patient having a more serious emergency may cause the wait time to be prolonged. Especially in the medical industry, such delays may cause further health complications for the patient, not only due to the symptoms of the original reason for seeking care, but also due to the stress of not knowing when care will be provided. Prolonged and unknown waiting times can create a negative environment within a healthcare facility, and can detract from the patient's recovery process.

Attempts have been made to provide more accurate waiting times, particularly in the food service industry, to improve customer satisfaction. By providing accurate waiting times, a customer can plan to arrive at a restaurant at a specific time, allowing the customer to more efficiently plan for activities before and after spending time at the restaurant. In addition, attempts have been made to create digital queues, such as in the retail industry. Customers can check into the digital queue and can explore different portions of a store while waiting to check out. Such digital queues not only improve store efficiency, but also improve customer satisfaction and can lead to increased sales.

Despite these attempts to improve the process of waiting for care or service, the above solutions simply provide a customer or a patient with a more accurate prediction of a waiting time. While the prediction may help alleviate some dissatisfaction, in certain situations, a customer or patient cannot spend time doing anything other than waiting. For example, a patient waiting to be seen in an emergency room may be unable to occupy prolonged waiting times with other activities. In these situations, predictions and updates to waiting times serve as simple status updates, and may not function to alleviate the patient's stress level. In addition, during the waiting time, a patient typically remains in the dark about his or her medical condition, the identity of the treating healthcare provider, and a potential treatment plan. Patients and their family members, friends, and loved ones would benefit from spending waiting times learning medical information that is pertinent to their individual needs.

Accordingly, what is needed is an interactive and dynamic mobile platform that not only predicts waiting times, but also triggers communications and content to be transmitted to an electronic device based on inputted and calculated data, thereby providing meaningful updates to the electronic device user and improving the user's waiting experience. Moreover, there is a need for such a mobile platform that does not add additional tasks to a physician's workflow, but instead maintains or reduces such tasks by improving the communication efficiencies involved in a medical care situation. However, in view of the art considered as a whole at the time the present invention was made, it was not obvious to those of ordinary skill in the field of this invention how the shortcomings of the prior art could be overcome.

### BRIEF SUMMARY OF THE INVENTION

The long-standing but heretofore unfulfilled need for a mobile application that dynamically transmits content and updates sent to electronic devices based on inputted and calculated data is now met by a new, useful, and nonobvious invention.

The novel method of determining and improving mobile application user satisfaction includes a step of receiving, on a mobile network, data from a plurality of electronic devices associated with a plurality of users. The mobile network also receives data from a facility in which the mobile network is utilized, such as the number of users, demographic information, staff information, and other facility-wide data. After receiving the relevant data, the mobile network transmits a first batch of information specific to each of the plurality of electronic devices, with the first batch of information being based on the data received by the mobile network.

The system determines an expected outcomes score for each of the plurality of users, with the expected outcomes score adapted to estimate user satisfaction for each of the plurality of users. The expected outcomes score is based on the data from the plurality of electronic devices associated with the plurality of users, as well as the data from the facility. The mobile network also receives updated data from the plurality of electronic devices associated with the plurality of users, as well as from the facility.

After a predetermined amount of time, the system measures an observed outcomes score for each of the plurality of users, which, similar to the expected outcomes score, is based on the updated data from the plurality of electronic devices associated with the plurality of users, as well as the updated data from the facility. For each individual user, the observed outcomes score is compared with the expected outcomes score to calculate an experience value index score, which is adapted to represent mobile application user satisfaction.

In an embodiment, if the experience value index score is above a threshold value for an individual user, the mobile network transmits a second batch of information to the user's electronic device, with the second batch of information including routine updates to the first batch of information. Conversely, if the experience value index score is below the threshold value, the mobile network transmits a third batch of information that includes interacted content designed to engage with the user and improve the user's satisfaction. In an embodiment, the system reevaluates the experience value index score after a predetermined amount of time by repeating the steps of determining the expected outcomes score, measuring the observed outcomes score, and calculating the experience value index score. A first weighted correlation may be calculated for one parameter, and a second weighted correlation may be calculated for a second parameter, with the weighted correlations being different from each other to provide more weight to one parameter during calculations. During the reevaluation, the weighted correlations may be updated depending on the experience value index score.

An object of the invention is to provide a mobile application that provides targeted information to application users based on the user's overall value index score, which is calculated by comparing expected results to actual results. Users having lower value index scores receive more in-depth and interactive batches of information in an attempt to increase their value index scores. Users having higher value index scores receive more routine batches of information due to their already-high value index scores. Accordingly, an object of the invention is to increase or maintain value index scores through targeted communications with application users.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1A depicts a screenshot from a mobile device according to an embodiment of the present invention, including a communication-based download link to a mobile application.

FIG. 1B depicts a screenshot from the mobile device of FIG. 1A, including a sign-in and authentication page.

FIG. 1C depicts a screenshot from the mobile device of FIG. 1A, including an inbox of real-time communications regarding a patient's status transmitted to the mobile device.

FIG. 1D a screenshot from the mobile device of FIG. 1A, including a continuation of the inbox showing continued real-time communications regarding a patient's status transmitted to the mobile device.

FIG. 1E depicts a screenshot from the mobile device of FIG. 1A, including graphical representations of clinic performance and activity as an interactive dashboard displayed on the mobile device via a mobile application.

FIG. 1F depicts a screenshot from the mobile device of FIG. 1A, including an inbox of real-time communications including educational information about the clinic and its policies.

FIG. 1G depicts a screenshot from the mobile device of FIG. 1A, including an informational page about a patient's healthcare team.

FIG. 2 depicts a flow diagram of an encryption system according to an embodiment of the present invention, including an authenticated log-in sequence, generation of an access token, and ensuring that a mobile device has an access token to log-in to the mobile application.

FIG. 3 depicts a feedback loop for measuring a patient experience value index score based on different factors, such as department capacity, patient information, engagement with the mobile application, and patient survey results.

FIG. 4 is a process flow diagram of a method of determining and reevaluating the patient experience value score of FIG. 3.

FIG. 5 is a process flow diagram of determining and reevaluating the patient experience value score of FIG. 3 by analyzing one particular metric-expected waiting time versus actual waiting time.

### DETAILED DESCRIPTION OF THE INVENTION

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

As used in this specification and the appended claims, the singular forms “a,” “an,” and “the” include plural referents unless the content clearly dictates otherwise; the term “or” is generally employed in its sense including “and/or” unless the context clearly dictates otherwise; and the terms “approximately” and “about” includes a recited value±ten percent unless the context clearly dictates otherwise.

The present invention includes a mobile application and methods of interacting with application users to transmit targeted, individually-curated information based on a user's needs and calculated experience value. The mobile application provides a transparent and secure platform through which authorized users can receive relevant information and provide feedback regarding satisfaction levels. The mobile application receives user feedback and determines the proper information to send to individual application users.

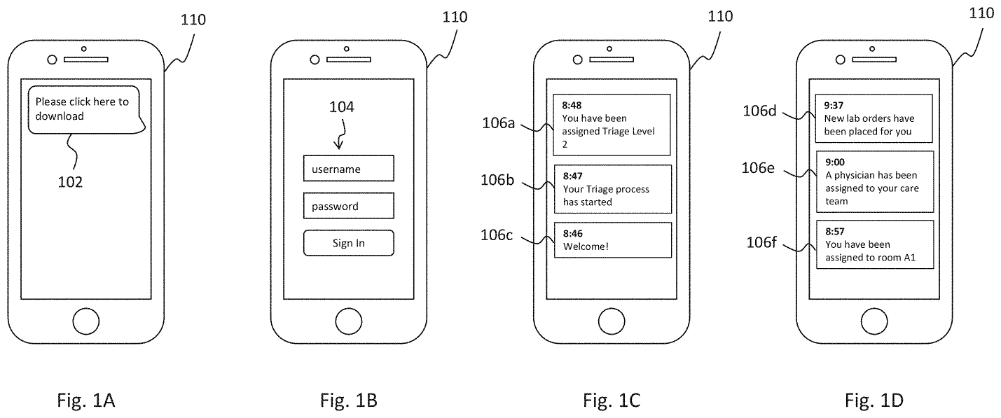

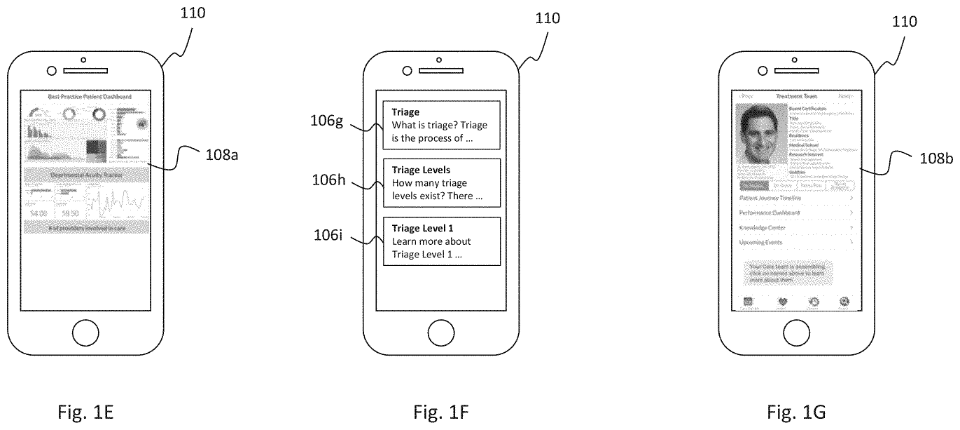

Referring to FIGS. 1A-1G, various screenshots of the mobile application are depicted. In the embodiment depicted in FIGS. 1A-1G, the mobile application is based in a healthcare facility, and application users are patients and their loved ones waiting to receive care. While the embodiment shown in FIGS. 1A-1G is an emergency room-based application, it is appreciated that the mobile application could be utilized in different industries in which users benefit from real-time, curated updates, including the food service, retail, and customer service industries.

The first screenshot shown in FIG. 1A includes mobile communication 102 transmitted by a real-time patient engagement system and displayed on electronic device 110. The real-time patient engagement system includes a mobile application that is interactable on a front-end by a user via electronic device 110, and a back-end network and server that communicates with the mobile application. As shown in FIG. 1A, the system transmits communication 102 from the back-end network to the front-end application, inviting a front-end user to interact with the mobile application on electronic device 110. For example, the system can send a communication via RF waves from an antenna. The communication is received by an application user's mobile device via an RF antenna connected to the device, thereby allowing the system to communicate with the mobile application user. Additional communications are depicted in FIGS. 1C-1D and 1F and denoted as reference numerals 106a-106i. It should be appreciated that the communications can be short message service messages, multimedia messaging services messages, push notifications, in-app messages, and other standard communication protocols.

As shown in FIG. 1B, the front-end user signs into the mobile application via a secure connection to ensure that the user has the proper permission to interact with the system. The secure connection appears on electronic device 110 as a login page 104, through which a user must enter credentials to gain access to the underlying application. Once the system ensures that the user is authorized to sign into the mobile application, the user can access and view the contents of the application. The system transmits real-time updates via an inbox that syncs with the mobile application, such that the user can view status updates, such as waiting times, triage status, caregiver assignments, and other useful information—as discussed above, these updates are shown in FIGS. 1C-1D and 1F, and denoted as reference numerals 106a-106i. In addition, as shown in FIGS. 1E and 1G, the system provides a transparent view of current healthcare facility statistics, general information about facility protocols and explanations of caregiving steps, and biographical information about healthcare professionals—this information is denoted as reference numerals 108a in FIG. 1E and 108b in FIG. 1G. This initial batch of information transmitted by the system to the mobile application is referred to as a first batch of relevant information.

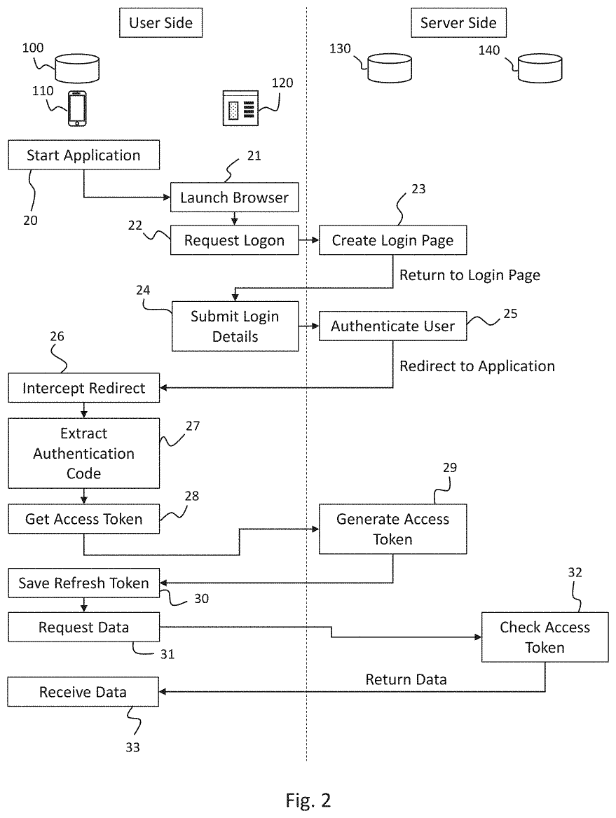

Turning now to FIG. 2, the encryption system is shown in greater detail. Regardless of the ultimate platform, privacy and security of user data is of utmost importance; however, an embodiment of the invention is utilized in the healthcare industry, which necessarily involves the flow of sensitive and private information, some of which is governed by strict privacy laws. As such, the system includes an authentication system that triggers after the user accesses the mobile application, with the system including a user side (depicted on the left side of the dashed line of FIG. 2) and a server side (depicted on the right side of the dashed line of FIG. 2). The user side includes electronic device 110 which is designed to execute files associated with the mobile application, embedded browser 120 which may be included within the mobile application or otherwise in communication with the mobile application; and application server 100 which is in communication with electronic device 110 and the servers on the server side of the dashed line. The server side includes authentication server 130 and resource server 140, each of which functions to verify a credential of a user of the mobile application executable on electronic device 110. The authentication steps are discussed herein below.

During step 20, the mobile application is executed on electronic device 110, which then launches browser 120 during step 21. When browser 120 is launched, during step 22, the system requests permission to logon to the mobile application, sending a request to authentication server 130. Authentication server 130 creates a login page during step 23, which then sends the login page to electronic device 110. During step 24, the user inputs and submits login details, with the login information being transmitted to authentication server 130. During step 25, authentication server 130 attempts to authenticate the user based on the login information. For example, authentication server 130 can verify the unique identifier information associated with electronic device 110 that is device-specific to authenticate the user. The system transmits the authentication request to a database (which may be stored on resource server 140) that includes the medical records and information of individual patients, the information database being separate from the system. Importantly, sensitive data is stored on the external, network-side information database, and not on local mobile application user devices-users can access data from the database, but that data remains stored and encrypted on the network side of the system. If successful, an authentication code is generated and sent to electronic device 110, and browser 120 redirects to the mobile application, which intercepts the redirect during step 26. During step 27, the mobile application extracts the authentication code from the information transmitted to the mobile application from authentication server 130 during the redirect.

Next, during step 28, electronic device 110 requests an access token that is usable for a predetermined amount of time, after which a new access token must be received (or the previous access token must be renewed). The authentication code extracted during step 27 is exchangeable for the access token requested during step 28. The request is received by authentication server 130, and an access token is generated during step 29. The access token is transmitted to electronic device 110 and is saved during step 30. The mobile application requests information or data from resource server 140. While the access token is active, the system retrieves requested information from the database to transmit the information to the user's device. In an embodiment, the system receives a refresh token in addition to the access token, with the refresh token being encrypted to prevent the vulnerability of the token. The refresh token is used to obtain new access tokens after the predetermined lifespan of an access token has expired.

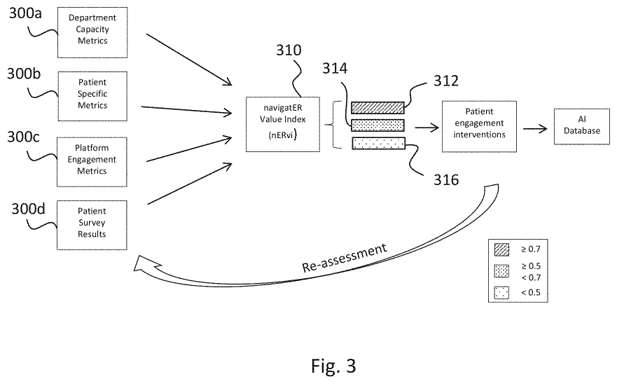

FIG. 3 provides an overview of one aspect of the system, the patient-specific engagement model. When a patient arrives at a healthcare facility, such as an emergency room, patient information is entered into an electronic medical record. The electronic medical record is stored in the information database discussed above. For example, the patient's condition and personal information, including demographic information, is stored within the information database. This process is repeated for each patient within a healthcare facility, thereby creating a database of individual patient records. The database is useful for medical professionals who can access the information stored therein; however, patients are often unable to access their electronic medical records in real-time in a traditional healthcare facility. Accordingly, according to an embodiment of the present invention, the system provides a medium through which the patient and his or her loved ones can interact with the patient's electronic medical record, such as through the authentication methods described above and shown in FIG. 2.

The system receives and stores information from different sources. Department capacity metrics 300a are provided by the information database, and the system utilizes these metrics to sort through critical information, such as the number of patients in a particular facility, the triage category of the patients, and the average time to a first evaluation (an average of the waiting time in the facility). In addition, the system analyzes patient arrival times, the day of the week, and the month of the year, to determine expected outcomes under the department capacity metrics 300a. Not only does the system receive facility-wide information, the system also receives patient-specific metrics 300b, such as the patient's age, sex, race, and injury status. The system utilizes all of this information to transmit a first batch of relevant information to the patient based on facility and patient-specific information, as discussed above.

After the patient first engages with the system, the system receives data from the patient's electronic device, including the time the patient spends on the platform compared with other mobile applications, and the time the patient spends on individual training materials on the mobile application. These metrics are received as platform engagement metrics 300c, and provide an insight into whether and how much patients interact with the mobile application during a visit to a care facility. If surveys are transmitted to the patient's mobile device, the system receives the results of the surveys as patient survey results 300d; alternatively, if the patient chooses to ignore a survey, the system receives data stating that the patient did not interact with a survey, also included in patient survey results 300d.

Still referring to FIG. 3, the department capacity metrics 300a, the patient specific metrics 300b, the platform engagement metrics 300c, and the patient survey results 300d are all utilized to determine a patient experience value index (or navigatER Value Index, nERvi, as denoted in FIG. 3) during step 310 of the process-flow diagram of FIG. 3. The patient experience value index is a ratio of two value scores—the expected outcomes value score, and the observed outcomes value score. The following formula (Equation (1)) is used to determine the patient experience value index:

$$\frac{observed outcomes value score}{expected outcomes value score} = patient experience value index \qquad ( 1 )$$

The expected outcomes value score is calculated based on the department capacity metrics 300a and patient specific metrics 300b—in other words, the system analyzes the then-current department and patient statistics, to determine an expected outcome for the patients in the healthcare facility, and calculate a score for the expected outcome. The expected outcomes value score is determined via an algorithm that determines an output value based on a set of input parameters (the weight of the input parameters can vary depending on the implementation of the system, such as data from the particular facility in which the system is employed). The department capacity input parameters include the total number of patients in the facility, the number of patients waiting to be admitted to the facility or discharged from the facility, the door-to-evaluation-time for patients sorted by triage category, and the time of the visit as a function of the time of year. The patient specific input parameters include the patient's age, sex, ethnicity, chief complaint, pain scores, vital signs, triage category, door-to-initial-evaluation time, evaluation location (such as a private room or a hallway bed), evaluation-to-disposition-decision time, disposition-decision-to-discharge time, and the patient's total length of stay.

Conversely, the observed outcomes value score is based on individual patient data—the platform engagement metrics 300c and the patient survey results 300d. Similar to the expected outcomes value score above, the observed outcomes value score is determined via an algorithm. For example, the system analyzes whether a patient spends time interacting with educational or training materials in the system, or whether the patient fails to interact with the system, to calculate a score for the observed outcome. The platform engagement input parameters include the total time a patient spends viewing and interacting with the mobile application, the amount of information and contents accessed by the patient, and the notifications sent to related application users, such as family and friends of the patient. The patent survey input parameters include whether the patient engaged with or ignored the survey, the completed survey results, and the evaluation times (such as whether patient expectations were met, exceeded, or missed). Similar to the then-current department and patient statistics discussed above, the weight of the individual patient data parameters can vary depending on the implementation of the system.

The system can be customized to provide different weights to different parameters, depending on some initial input information. For example, in a first step, the system assesses the relationship between outcome (dependent) variables and potential predictor (independent) variables. During this step, the system accounts for historical data for an individual institution—for example, for a first institution, the strongest variables for predicting an outcome may be patient age, triage acuity, the number of available waiting rooms during intake, and the number of patients in the waiting room during intake. For a second institution, the strongest variables may be the patient's total length of stay, interaction with the mobile application, and survey results. The system determines the strength and direction of the relationships between outcome variables and predictor variables by utilizing Equation (2):

$$r = \frac{{cov}_{xy}}{{s}_{x} {s}_{y}} = \frac{{\sum }_{i = 1}^{n} ( {x}_{i} - \overline{x} ) ( {y}_{i} - \overline{y} )}{( N - 1 ) {s}_{x} {s}_{y}} , \qquad ( 2 )$$

where x̅ is the mean of the sample independent variable, xᵢ is the independent variable data point of interest, yᵢ is the corresponding dependent variable value, y̅ is the mean of the sample dependent variable, N is the number of observations, sₓ is the standard deviation of the predictor (independent) variable and s_{y} is the standard deviation of the outcome (dependent) variable. Equation (2) can be repeated for each measured variable, and the results compared to determine the prediction variables having the strongest correlation with the outcome variables.

The result of Equation (2) can be used to create a robust, multivariate model that predicts outcomes of interest. The strongest predictor variables identified in Equation (2) are advanced as candidates for inclusion in multivariate models using Equation (3):

Yᵢ=(b₀+b₁X₁ᵢ+b₂X₂ᵢ+b₃X₃ᵢ+ . . . bₙXₙᵢ)+εᵢ (3)

where Y is the outcome variable of interest, b₁ is the coefficient of the first predictor (Xᵢ), b₂ is the coefficient of the second predictor (X₂), b₃ is the coefficient of the third predictor (X₃), bₙ is the coefficient of the nth predictor (Xₙᵢ) and εᵢ is the error for the ith participant. Equation (3) illustrates that as many predictor variables as necessary can be added until the final predictor, Xₙ, is determined, with each predictor being assigned a regression coefficient (b). The number of variables, as well as the specific predictors, may vary between institutions. The prediction process is iterative and information regarding the accuracy of early predictions may be added as a potential predictor to later iterations, allowing for improved predictive accuracy and reducing the model error component.

The system uses all of this information to calculate the patient experience value index, which is on a 0.0-1.0 scale. In addition, the system separates the patient experience value index scores into different categories. In an embodiment, three categories are employed, as depicted in FIG. 3—a slanted line pattern (denoted by reference numeral 312), representing a value index greater than or equal to 0.7; a clustered dot pattern (denoted by reference numeral 314), representing a value index greater than or equal to 0.5 and less than 0.7; and a sporadic dot pattern (denoted by reference numeral 316), representing a value index less than 0.5. As alluded to above, a higher value index score indicates a higher patient satisfaction, because the observed outcome value score was close to the expected value score. Conversely, a lower value index score indicates a lower patient satisfaction, because the expected outcomes did not occur.

After calculating the patient experience value index scores, the system transmits a second batch of relevant information to each individual user based on the individual user's value scores. For example, the system may transmit routine updates to a user with a high value index score, whereas the system transmits targeted, personalized, and interactive engagement tools to a user with a low value index score. As such, the system attempts to engage with users having low value index score to improve their scores, such as by obtaining and transmitting interactive materials from a database based on scores, patient metrics, and learned patterns. Importantly, the relevant information transmitted to users is individualized to patient information, including patient condition and triage category—as such, the information sent to users differs based on patient condition, as well as value index scores. After sending the second batch of relevant information, the system reassesses, or reevaluates, the patient experience value index for individual users. The system steps are discussed herein below.

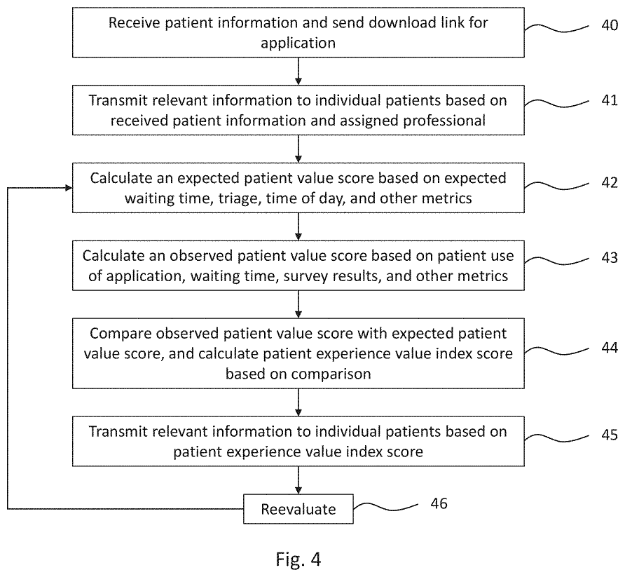

FIG. 4 is a process flow diagram that outlines an embodiment of the system. During step 40, the system receives patient information, such as the patient's identification number (typically a social security number), name, and electronic device contact information (i.e., a telephone number or email address). The system transmits a download link to the patient's electronic device, which the patient uses to gain access to the system and to the patient's electronic medical records (as discussed in detail above, FIG. 2 and its associated detailed description outlines an embodiment of the authentication steps taken by the system to ensure the confidentiality of information transmitted by and within the system). In step 41, after the patient logs into the system, the system retrieves relevant information that is personalized for the individual patient, and transmits such information to the patient. Examples of relevant information are the patient's triage category, assigned caregiving team, educational content on the patient's condition, the healthcare facility, the treating physician, and similar information.

During step 42, the system calculates an expected patient value score, as discussed above. After a predetermined amount of time, during step 43, the system calculates an observed patient value score, again as discussed above. During step 44, the system compares these value scores to calculate the patient experience value index score discussed above, and during step 45, the system again transmits relevant information to individual patients based on the experience value index scores. During step 46, the system reevaluates the patient experience value index score by recalculating expected and observed patient value scores.

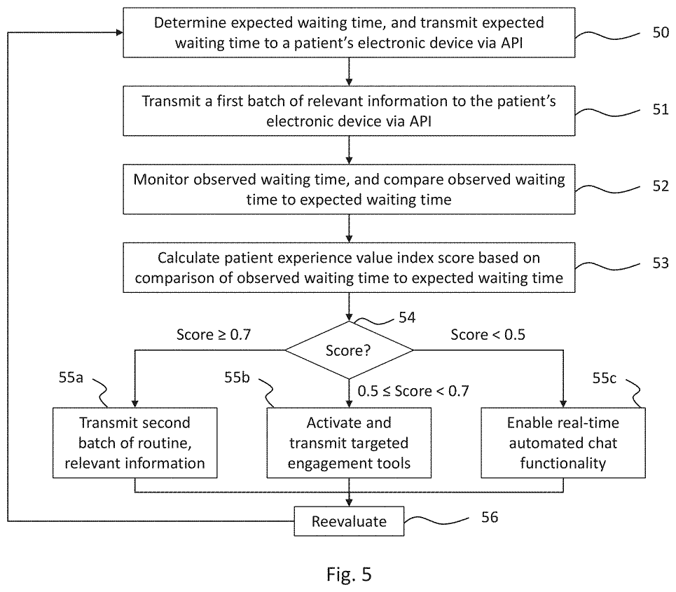

FIG. 5 is a process flow diagram of one particular aspect of the system, the patient experience value index score as determined by a singular factor, patient waiting time. During step 50, the system determines an expected waiting time based on facility-specific factors and the patient's triage category. The system transmits the expected waiting time to the patient's electronic device via an API. During step 51, the system transmits a first batch of relevant information to the patient's device via an API, as discussed in detail above. During step 52, the system monitors the observed waiting time of individual patients, and compares the observed waiting times to the expected waiting times for all patients in a facility at a given time. During step 53, the system utilizes the comparison between the observed and expected waiting times to generate a patient experience value score.

Step 54 depicts the different score thresholds used in an embodiment of the system-if the score is greater than or equal to 0.7 (on a scale of 0.0-1.0, as discussed above), then the system triggers step 55a, during which the system transmits a second batch of routine, relevant information to the individual patients having associated high scores. If the score is greater than or equal to 0.5 but less than 0.7, the system triggers step 55b, during which the system activates and transmits targeted engagement tools designed to help improve patient satisfaction despite a longer-than-expected waiting time. If the score is below 0.5, the system triggers step 55c, during which the system enables a real-time, automated chat functionality. During step 55c, the system communicates with individual patients having associated low scores, in an attempt to engage with and improve patient satisfaction. During step 56, the system reevaluates the patient experience value index score by determining a new expected waiting time, and proceeding through the steps outlined above.

The steps delineated in the process-flow diagrams of FIGS. 4-5 are merely exemplary of orders of calculating and reevaluating patient satisfaction scores. The steps may be carried out in another order, with or without additional steps included therein.

### Glossary of Claim Terms

Communication: is a request, alert, report, or message, whether sent or received via text or voice, by users of a mobile application, with the communication sent via the mobile application as an intermediary that wirelessly receives and sends transmissions.

Electronic device: is a cellular phone, tablet, computer, or other electronic device with networking capabilities, that can execute the instructions of a mobile application.

Information: is a communication including a set of data transmitted by a mobile application to a user's electronic device. Examples of information include status updates, waiting time, educational content, interactive surveys, and other similar communications.

Mobile application: is a software program or system designed to function on one or more mobile devices and mobile networks.

Mobile network: is a communication network involving mobile devices, which allows communication through a mobile application.

User: is a user of a mobile application who receives communications from a mobile network via the mobile application.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A method of determining mobile application user satisfaction, the method comprising the steps of:
receiving on a mobile network data from a plurality of electronic devices associated with a plurality of users;
receiving on the mobile network data from a facility in which the mobile network is utilized;
transmitting from the mobile network to each of the plurality of electronic devices a batch of information individually selected based on the data received from the plurality of users and from the facility;
determining an expected outcomes score for each of the plurality of users, the expected outcomes score based on the data from the plurality of electronic devices associated with the plurality of users, and the data from the facility, the expected outcomes score adapted to estimate user satisfaction for each of the plurality of users;
receiving on the mobile network updated data from the plurality of electronic devices associated with the plurality of users, and updated data from the facility in which the mobile network is utilized;
after a predetermined amount of time, measuring an observed outcomes score for each of the plurality of users, the observed outcomes score based on the updated data from the plurality of electronic devices associated with the plurality of users, and the updated data from the facility; and
for each of the plurality of users, comparing the observed outcomes score with the expected outcomes score to calculate an experience value index score, the experience value index score adapted to represent mobile application user satisfaction.

2. The method of claim 1, wherein the data received from the plurality of users includes the total time spent viewing and interacting with the batch of information.

3. The method of claim 1, wherein the data received from the facility for at least one of the plurality of users includes at least one parameter selected from the group consisting of the user's age, sex, ethnicity, injury, pain score, vital level, triage category, door-to-initial-evaluation time, evaluation location, and total length of stay.

4. The method of claim 1, wherein the data received from the facility includes at least one parameter selected from the group consisting of a number of patients waiting to be admitted to the facility, a number of patients waiting to be discharged from the facility, a door-to-evaluation-time for patients sorted by triage category, and a time of a visit as a function of a time of year.

5. The method of claim 1, further comprising a step of transmitting from the mobile network to each of the plurality of electronic devices an updated batch of information individually selected based on the calculated experience value index score.

6. The method of claim 5, wherein the updated batch of information for at least one of plurality of users includes a patient survey.

7. The method of claim 6, wherein answers to the patient survey and engagement with the patient survey are used to update the observed outcomes score.

8. The method of claim 1, wherein the step of determining the expected outcomes score for each of the plurality of users further comprises:
calculating a first weighted correlation for at least one parameter of the data from the plurality of electronic devices associated with the plurality of users; and
calculating a second weighted correlation for at least one parameter of the data from the facility,
wherein the first weighted correlation is different from the second weighted correlation.

9. The method of claim 8, further comprising a step of recalculating the expected outcomes score for each of the plurality of users by updating at least one of the first weighted correlation and the second weighted correlation.

10. A method of determining mobile application user satisfaction, the method comprising the steps of:
receiving on a mobile network data from an electronic device associated with a user;
receiving on the mobile network data from a facility in which the mobile network is utilized;
transmitting from the mobile network to the electronic device a batch of information individually selected based on the data received from the user and from the facility;
determining an expected outcomes score for the user, the expected outcomes score based on the data from the electronic device associated with the user and the data from the facility, the expected outcomes score adapted to estimate user satisfaction for the user;
receiving on the mobile network updated data from the electronic device associated with the user and updated data from the facility in which the mobile network is utilized;
after a predetermined amount of time, measuring an observed outcomes score for user, the observed outcomes score based on the updated data from the electronic device associated with the user, and the updated data from the facility;
comparing the observed outcomes score with the expected outcomes score to calculate an experience value index score, the experience value index score adapted to represent mobile application user satisfaction for the user.

11. The method of claim 10, wherein the data received from the user includes the total time spent viewing and interacting with the batch of information.

12. The method of claim 10, wherein the data received from the facility for the user includes at least one parameter selected from the group consisting of the user's age, sex, ethnicity, injury, pain score, vital level, triage category, door-to-initial-evaluation time, evaluation location, and total length of stay.

13. The method of claim 10, wherein the data received from the facility includes at least one parameter selected from the group consisting of a number of patients waiting to be admitted to the facility, a number of patients waiting to be discharged from the facility, a door-to-evaluation-time for patients sorted by triage category, and a time of a visit as a function of a time of year.

14. The method of claim 10, further comprising a step of transmitting from the mobile network to the electronic device an updated batch of information individually selected based on the calculated experience value index score.

15. The method of claim 14, wherein the updated batch of information for the user includes a patient survey, and wherein answers to the patient survey and engagement with the patient survey are used to update the observed outcomes score.

16. The method of claim 10, wherein the step of determining an expected outcomes score for the user further comprises the steps of:
calculating a first weighted correlation for at least one parameter of the data from the electronic device associated with the user; and
calculating a second weighted correlation for at least one parameter of the data from the facility,
wherein the first weighted correlation is different from the second weighted correlation.

17. The method of claim 16, further comprising a step of recalculating the expected outcomes score for the user by updating at least one of the first weighted correlation and the second weighted correlation.

18. A method of improving mobile application user satisfaction, the method comprising the steps of:
receiving on a mobile network data from a plurality of electronic devices associated with a plurality of users, and data from a facility in which the mobile network is utilized;
transmitting from the mobile network to each of the plurality of electronic devices a first batch of information individually selected based on the data received from the plurality of users and from the facility;
determining an expected outcomes score for each of the plurality of users, the expected outcomes score based on the data from the plurality of electronic devices associated with the plurality of users, and the data from the facility, the expected outcomes score adapted to estimate user satisfaction for each of the plurality of users;
receiving on the mobile network updated data from the plurality of electronic devices associated with the plurality of users, and updated data from the facility in which the mobile network is utilized, wherein the updated data from each of the plurality of electronic devices associated with each of the plurality of users includes information about individual user interaction with the first batch of information;
after a first predetermined amount of time, measuring an observed outcomes score for each of the plurality of users, the observed outcomes score based on the updated data from the plurality of electronic devices associated with the plurality of users, and the updated data from the facility;
for each of the plurality of users, comparing the observed outcomes score with the expected outcomes score to calculate an experience value index score, the experience value index score adapted to represent mobile application user satisfaction;
for each of the plurality of users, if the experience value index score is above a threshold value, transmitting from the mobile network to the electronic device associated with the user a second batch of information, the second batch of information including updates to the first batch of information;
for each of the plurality of users, if the experience value index score is below the threshold value, transmitting from the mobile network to the electronic device associated with the user a third batch of information, the third batch of information including interactive content designed to engage with the user; and
after a second predetermined amount of time, reevaluating the experience value index score by re-determining the expected outcomes score, re-measuring the observed outcomes score, and comparing the re-measured observed outcomes score to the re-determined expected outcomes score.

19. The method of claim 18, wherein the step of determining the expected outcomes score for each of the plurality of users further comprises the steps of:
calculating a first weighted correlation for at least one parameter of the data from the plurality of electronic devices associated with the plurality of users; and
calculating a second weighted correlation for at least one parameter of the data from the facility,
wherein the first weighted correlation is different from the second weighted correlation.

20. The method of claim 19, wherein the step of re-determining the expected outcomes score further comprises updating at least one of the first weighted correlation and the second weighted correlation.
